(19) United States
(12) Patent Application Publication
(10) Pub. No.: US 2021/0007688 A1
(43) Pub. Date: Jan. 14, 2021

(54) ROBOTICALLY POSITIONED X-RAY AND C-ARM

(71) Applicant: Peter L. Bono, (US)

(72) Inventors: Peter L. Bono, Bingham Farms, MI (US); James D. Lark, West Bloomfield, MI (US)

(21) Appl. No.: 16/906,159

(22) Filed: Jun. 19, 2020

Related U.S. Application Data
(60) Provisional application No. 62/864,269, filed on Jun. 20, 2019.

(51) Int. Cl. A61B 6/00; A61B 34/30; A61B 34/20; A61B 90/00

(52) U.S. Cl. CPC A61B 6/4458; A61B 6/54; A61B 6/4441; A61B 34/30; A61B 2034/2055; A61B 90/36; A61B 2090/376; A61B 2034/2051; A61B 34/20

(57) ABSTRACT

The invention involves a system and method for taking x-rays in a surgical setting. The system includes a multi-axis robot having an automatic tool change system. The robot can therefore go to the tools and pick up the x-ray apparatus, which may be in the form of a C-arm. The x-ray apparatus can then be manipulated about the patient to obtain the desired x-rays, which are viewable in real time. The x-ray apparatus can then be repositioned in the tool rack and the robot utilized for other parts of the surgery. Should additional x-rays be needed, the robot can reattach the x-ray apparatus and position the apparatus according to a new location; or the robot can track any movements of the patient via an electromagnetic or radio frequency sensor system, whereby the x-ray apparatus can be positioned exactly as it was with respect to the patient from a previous x-ray.

### RELATED APPLICATIONS

In accordance with 37 C.F.R 1.76, a claim of priority is included in an Application Data Sheet filed concurrently herewith. Accordingly, the present invention claims priority to U.S. Provisional Patent Application No. 62/864,269, filed Jun. 20, 2019, entitled, “Robotically Positioned X-Ray and C-Arm”, the contents of which are incorporated herein by reference. This application is also related to co-pending U.S. patent application Ser. No. 16/246,291, filed Jan. 11, 2019, entitled “Surgical Sensor Anchor System”, which is a continuation-in-part of U.S. patent application Ser. No. 15/816,861, filed Nov. 17, 2017, entitled “Robotic Surgical System,” which claims priority to U.S. Provisional Patent Application No. 62/423,677, filed Nov. 17, 2016, entitled “Robotic Surgical System”.

### FIELD OF THE INVENTION

The present invention generally relates to medical imaging; and more particularly, to an imaging arm constructed for positioning on the distal end of an automotive style robotic arm constructed to track movements of the patient for image repeatability.

### BACKGROUND INFORMATION

Fluoroscopy machines are often used in hospital emergency rooms and trauma centers. These machines have an arm which supports an x-ray source spaced apart from an x-ray detector. The arm, generally a C-shaped arm, is utilized to locate the x-ray source with respect to the x-ray detection; and can be manipulated to place the x-ray source on one side of a patient and the x-ray detector on the other side of the patient. A series of joints permit the arm to be manually moved to a pose which will provide a desired x-ray image. A monitor displays the x-ray image in real time. C-arm fluoroscopy machines may, for example, be used to image the locations at which pins or screws will be inserted to hold bones in position.

One issue with the use of C-arm fluoroscopy machines is limiting the amount of x-rays to which physicians and other medical personnel are exposed. In many procedures, a physician's hands will be in the field irradiated by x-rays. Although modern x-ray machines can acquire acceptable images with a lower dose than was formerly possible, there is a limit to the dose reduction that can be achieved by this route.

Another approach to reducing x-ray exposure to medical personnel is reducing the amount of time required to obtain desired images by providing a system to track the position of the x-ray arm with respect to the patient's anatomy to reduce the time taken to obtain desired images.

Optical localizers have been proposed for tracking the position of the arms of C-arm fluoroscopy machines. Such localizers use cameras to track the position of targets mounted on the C-arm. Optical trackers have a number of deficiencies. For example, the camera requires an unobstructed line of sight to the targets. This constrains the use of valuable operating room space. This problem is made worse because a C-arm is relatively large and must be able to be moved through a large range of motion.

An additional drawback to these prior art systems relates to an inability to track the patient for movement. Even if the C-arm is moved to the position of a previous x-ray, positioning the device in the same position at a later point in a surgical procedure will unlikely result in the same x-ray due to movement of the patient.

Finally, there are ergonomic needs that an x-ray system must satisfy in order to achieve acceptance by the end user. The system must be easily and quickly positioned using minimal hardware and requiring a minimal number of tools. Further, the system should not require excessive strength to maneuver or include heavy component parts. Moreover, the system must assemble together in such a way so as not to detract from the ability of the surgical team to accomplish their desired task.

Thus, the present invention provides a robotically positioned x-ray device which may include a C-arm that overcomes the disadvantages of prior art x-ray systems. The x-ray system of the present invention provides for relative ease in the positioning by attaching the x-ray device to the distal end of a multi-axis automotive style robot. The system also permits tracking the patient's movements, and can therefore duplicate previous x-ray illustrations by positioning the x-ray equipment according to the patient and not a set of previous angular measurements. The present system also provides the X-ray system as part of a set of tools that can be attached to a robot as needed, or detached and stored for the next use.

### SUMMARY OF THE INVENTION

Briefly, the invention involves a system and method for taking x-rays in a surgical setting. In at least one embodiment, the system includes a multi-axis robot having an automatic tool change system. The robot can therefore go to the tools and pick up the x-ray apparatus, which may be in the form of a C-arm. The x-ray apparatus can then be manipulated about the patient to obtain the desired x-rays, which are viewable in real time. The x-ray apparatus can then be repositioned in the tool rack and the robot utilized for other parts of the surgery. Should additional x-rays be needed, the robot can reattach the x-ray apparatus and position the apparatus according to a new location; or the robot can track any movements of the patient via an electromagnetic or radio frequency sensor system, whereby the x-ray apparatus can be positioned exactly as it was with respect to the patient from a previous x-ray. The system can be operated with one or more robots that are in communication with each other electronically so that each robot knows the position and movements of the other in order to prevent interference or collusion between the robots.

Accordingly, it is an objective of the present invention to provide a robotically manipulated x-ray system for surgical procedures.

It is a further objective of the present invention to provide a robotically manipulated x-ray system that is connected to an automotive style robot.

It is yet a further objective of the present invention to provide a robotically manipulated x-ray system that is quickly attached and detached from the robot so that the robot can complete portions of the surgical procedure.

It is another objective of the instant invention to provide a robotically manipulated x-ray system that tracks the patient for movement to position and reposition the x-ray equipment.

Other objectives and advantages of this invention will become apparent from the following description taken in conjunction with the accompanying drawings wherein are set forth, by way of illustration and example, certain embodiments of this invention. The drawings constitute a part of this specification, include exemplary embodiments of the present invention, and illustrate various objects and features thereof.

### BRIEF DESCRIPTION OF THE FIGURES

FIG. 1 is a perspective view of one embodiment of the present system;

FIG. 2 is an end view of an alternative embodiment of the present system;

FIG. 3 is another alternative embodiment of the present system;

FIG. 4 is an end view of the embodiment illustrated in FIG. 1; and

FIG. 5 is yet another alternative embodiment.

### DETAILED DESCRIPTION OF THE PREFERRED EMBODIMENTS

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiments illustrated.

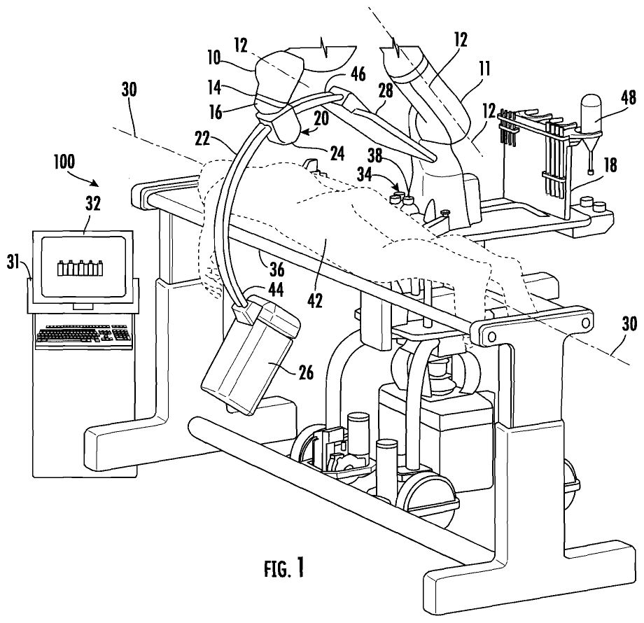

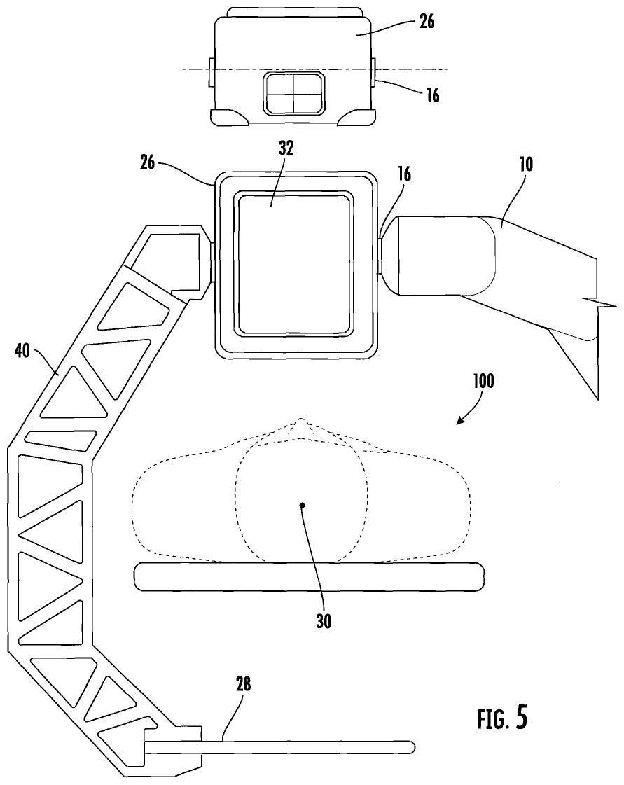

Referring generally to FIGS. 1-5, a robotically controlled X-ray system 100 for surgical procedures is illustrated. The system preferably includes a multi-axis computer controlled robot, preferably an automotive style robot 10. The robot includes multiple axes 12 about which portions of the robot rotate with respect to an adjacent portion of the robot 10 to position the distal end 14 of the robot arm assembly 11. The robot arm 11 may include up to seven axes of movement to allow the arm to be positioned as desired by the surgeon. The axes are typically monitored for position by encoders or the like, and provide feedback to the control computer 31 for precise control of the electric, hydraulic or pneumatic actuators utilized to provide movement to the robot arm. The distal end 14 of the robotic arm 11 also preferably includes a tool change coupler 16 that allows the robot to be quickly uncoupled from one tool 48 and coupled to a variety of alternate tools 48. The tools 48 are stored in a tool rack 18 in a manner that allows any one of the tools 48 to be attached to the distal end 14 of the robot 10 as desired. In operation, the robot 10 would automatically or on-command maneuver to the tool rack 18 to drop one tool into a desired position in the tool rack, and then move to the position of the second tool positioned in the tool rack to grasp the tool with the tool change coupler 16. The tool can then be moved and positioned as desired by the robot 10 through the robotic control computer 31. The x-ray arm 20 may include one or more C-shaped rails 22, a rail motor 24, an x-ray source 26 and an image receiver 28. The x-ray arm 20 includes a first end 44 having the x-ray source 26 secured thereto and a second end 46 having the image receiver 28 secured thereto so that the x-ray source and the image receiver may be positioned on opposite sides of an animal, preferably a human. The x-ray arm 20 is connected to the robot 10 in a manner that allows the rail motor 24 to traverse the C-shaped rail 22 in both directions with respect to the distal end 14 of the robot 10 as needed. This construction allows the x-ray source 26 and the image receiver 28 to be positioned around a longitudinal axis 30 of the patient to direct the x-rays through the patient 42 to the image receiver 28 from any angle along the longitudinal axis 30. Movement is also provided by the robot, which can angle the C-shaped rails 22 with respect to or move them anywhere along, closer to or farther away from the patient 42. It should also be noted that two robots 10 may be utilized without departing from the scope of the invention by using a first robot to manipulate the x-ray source 26 and a second robot to manipulate the image receiver 28. Images received by the image receiver 28 can then be displayed on a monitor 32 in real time and stored on any suitable storage medium suitable for storage of digital data, such as an image.

Still referring to the Figures, the robotically controlled x-ray system 100 also preferably includes a tracking system 34 for tracking movements of the patient during the procedure. Patients often move during a procedure from coughing, manipulating the body for the procedure, etc. The present system, therefore, preferably includes an electromagnetic, radio frequency or optic sensor system which can be connected to the patient, particularly the skeletal system, and in electrical communication and monitored by the robotically controlled x-ray system 100 to monitor these movements so that the x-ray system can duplicate previous images taken or position the x-ray system in any predetermined position determined by the surgeon. The monitoring system receiver 38 preferably monitors all three axes of movement to provide feedback from at least one sensor 36 to the receiver 38 in a wireless or wired manner. The X-ray system 100 can then compensate for these movements, even if the patient is moved several inches or rolled over. In some embodiments, more than one sensor and receiver can be utilized to prevent blind spots caused by equipment or persons in the operating room. Sensor and receiver systems suitable for use with the present system are disclosed in co-pending patent application Ser. No. 16/246,291, filed Jan. 11, 2019 entitled, “SURGICAL SENSOR ANCHOR SYSTEM”, the contents of which are incorporated herein by reference.

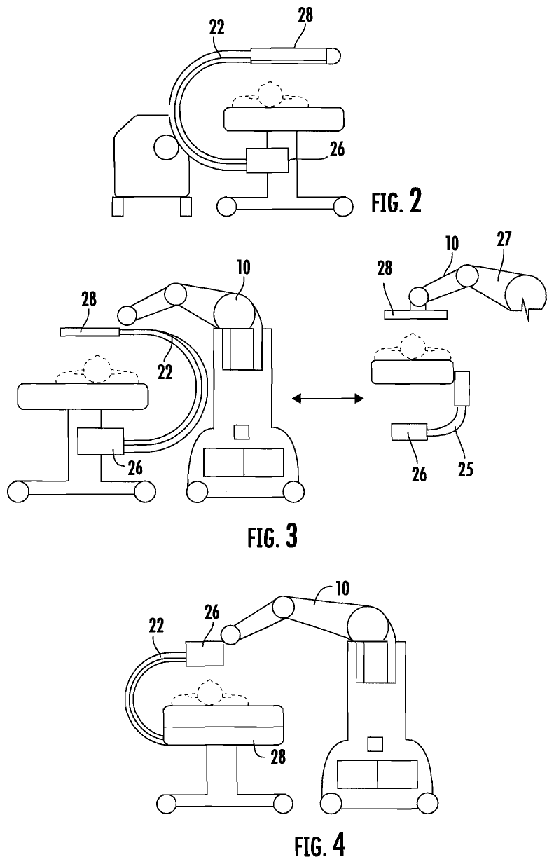

Referring to the Figures, and more particularly to FIG. 5, one embodiment of the robotically controlled x-ray system 100 includes an x-ray source 26 that is directly connected to the tool change coupler 16. In this embodiment, an arm 40 is rotationally secured to an opposite side of the X-ray source 26 and extends around the patient to retain the image receiver 28. FIG. 3 illustrates an embodiment having the x-ray source 26 connected to a first robotic arm 25, while the image receiver 28 is connected to a second robotic arm 27. Both the first and the second robotic arms may include one or more computer controlled axes of movement.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objects and obtain the ends and advantages mentioned, as well as those inherent therein. Any compounds, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A robotically controlled x-ray (100) system comprising:
a robot (10) having an arm assembly (11), said arm assembly having at least two axes of computer controlled movement for positioning a distal end (14) of said arm assembly (11) at a desired position and orientation in response to electric signals received from a control computer (31), an x-ray source (26) secured to said distal end (14) of said arm assembly (11) and an image receiver (28) secured at a position to receive x-rays from said x-ray source (26) to produce an image.

2. The robotically controlled x-ray system (100) of claim 1 wherein said arm assembly (11) includes up to seven axes of movement to allow said arm assembly (11) to be positioned as desired by a surgeon.

3. The robotically controlled x-ray system (100) of claim 1 wherein said axes of movement are monitored for relative position with respect to an adjacent axis of movement to provide electrical feedback to said control computer (31).

4. The robotically controlled x-ray system (100) of claim 1 wherein said x-ray source (26) includes an x-ray arm (20), said x-ray arm (20) including one or more C-shaped rails (22), said x-ray source (26) secured to a first end (44) of said one or more C-shaped rails (22) and an image receiver (28) secured to a second end (46) of said one or more C-shaped rails (22) to receive x-rays from said x-ray source (26) to produce an x-ray image on a monitor (32), said x-ray source (26) and said image receiver (28) positioned on said one or more C-shaped rails (22) to allow said x-ray source (26) and said image receiver (28) to be positioned on opposite sides of a patient (42).

5. The robotically controlled x-ray system (100) of claim 4 wherein said x-ray arm (20) includes a rail motor (24) for traversing said one or more C-shaped rails (22) in either direction along the longitudinal centerline of said one or more C-shaped rails (22).

6. The robotically controlled x-ray system (100) of claim 4 wherein said one or more C-shaped rails (22) are connected to said arm assembly (11) in a manner which allows said C-shaped rails (22) to be angled with respect to a longitudinal axis (30) of said patient (42).

7. The robotically controlled x-ray system (100) of claim 1 including a tracking system (34) for tracking movements of said animal, said tracking system (34) providing position feedback to said control computer (31), said control computer (31) constructed and arranged to utilize patient position to provide predetermined x-ray images.

8. The robotically controlled x-ray system (100) of claim 7 wherein said tracking system (34) utilizes electromagnetic sensors (36) to monitor said patient position.

9. The robotically controlled x-ray system (100) of claim 7 wherein said tracking system utilizes optic sensors (36) to monitor said patient position.

10. The robotically controlled x-ray system (100) of claim 7 wherein said tracking system (34) utilizes a combination of optic sensors and electromagnetic sensors (36) to monitor said patient position.

11. The robotically controlled x-ray system (100) of claim 1 wherein said distal end (14) of said arm assembly (11) includes a tool change coupler (16), said tool change coupler (16) constructed and arranged to selective coupling and uncoupling of said distal end (14) of said arm assembly (11) to one or more different tools (48).

12. The robotically controlled x-ray system (100) of claim 11 including a tool rack (18) positioned within reach and at a known position with respect to said distal end (14) of said arm assembly (11).

13. The robotically controlled x-ray system (100) of claim 12 wherein said tools (48) are stored in said tool rack (18) in a manner that allows any one of said tools (48) to be selectively attached to said distal end (14) of said arm assembly (11).

14. The robotically controlled x-ray system (100) of claim 13 wherein said tool rack (18) is constructed and arranged to allow said arm assembly (11) to move to a first known position adjacent said tool rack (18) for dropping a first tool (48) and wherein said arm assembly (11) is repositioned by said control computer (31) to position said arm assembly (11) in a second position adjacent said tool rack (18) to attach said distal end (14) of said arm assembly (11) to a second tool (48) supported by said tool rack (18).

15. The robotically controlled x-ray system (100) of claim 1 including a second robot (10) having a second arm assembly (11), said second arm assembly (11) having at least two axes of computer controlled movement for positioning a distal end (14) of said second arm (11) at a desired position and orientation in response to electric signals received from a control computer (31), image receiver (28) secured to said distal end (14) of said second arm assembly (11), said second arm assembly computer controlled to position said image receiver (28) at a position with respect to said arm assembly (11) to receive x-rays from said x-ray source (26) to produce an image.

16. The robotically controlled x-ray system (100) of claim 3 wherein said control computer (31) includes a storage medium for storing images received by said image receiver (28), said images being recallable for viewing.
